(19) United States
(12) Patent Application Publication
(10) Pub. No.: US 2026/0266790 A1
(43) Pub. Date: Sep. 10, 2026

(54) EVALUATION SYSTEM OF OPERATION

(71) Applicant: HITACHI HIGH-TECH CORPORATION, Minato-ku, Tokyo (JP)

(72) Inventors: Katsutoshi MURAMATSU, Tokyo (JP); Nobuaki NAKASU, Tokyo (JP); Kohei SATO, Tokyo (JP)

(21) Appl. No.: 19/164,357

(22) PCT Filed: Feb. 9, 2024
(86) PCT No.: PCT/JP2024/004476

§ 371 Date: Sep. 11, 2025

(51) Int. Cl. G01N 33/00; B23P 19/04

(52) U.S. Cl. CPC G01N 33/0078; B23P 19/04

(57) ABSTRACT

An evaluation system of an operation including a plurality of steps on an object having a surface for which a predetermined level of cleanliness is required, in which the evaluation includes calculating a total sum of a sum of a plurality of operation times required for the respective plurality of steps, and a product of the sum of the operation times and a coefficient obtained in advance indicating a degree of an occurrence of a defect due to a foreign matter deteriorating the level of cleanliness of the surface in the plurality of steps after the operation based on an evaluation result on a degree of diffusion or adhesion of the foreign matter, and the calculated result is displayed or notified on a display.

### TECHNICAL FIELD

The present invention relates to an evaluation system of an operation.

### BACKGROUND ART

In a semiconductor manufacturing device, a foreign matter generated in a manufacturing process adheres to an in-device part to deteriorate a yield, and thus a maintenance operation for periodically disassembling and cleaning the device is required. Since the maintenance operation is performed by stopping the device, there is a problem that a stop time is long and an operation rate is reduced in an equipment design or an operation procedure in which disassembly, cleaning, and assembly are difficult to perform.

Therefore, as a technique for creating an operation procedure for minimizing an operation time, a technique for estimating a time required for disassembly and assembly based on equipment design information and an operation procedure is disclosed. Since a disassembly operation and an assembly operation are symmetrical, the following description focuses on the assembly operation alone.

As a related art related to an assembly operation, PTL 1 aims to provide a program capable of improving an accuracy of an estimated value of an assembly time calculated in a simulation, and discloses the following contents as a program, an assembly time calculation method, and an assembly time calculation device: “a program for calculating an assembly time of a product by a simulation, in which an input unit 1302 acquires animation data for displaying a procedure of an assembling operation of the product on a display unit with an animation, a change point detection unit 1310 detects a change in a viewpoint of the animation from the acquired animation data, and a standard time calculation unit 1311 calculates an estimated value of the assembly time of the product based on the detected change in the viewpoint of the animation”. PTL 1 discloses a technique of estimating an assembly operation time by simulating an assembly operation in a computer using an assembly operation procedure of a product as an input.

PTL 2 aims to provide a device for evaluating and calculating assembly operability without the need for an evaluator to arrange and operate a human body model, and discloses the following contents as an assembly operability evaluation calculation device and an assembly operability evaluation method, “including an information acquisition unit that extracts, from a 3D CAD model, information on part attributes of a plurality of parts, part arrangements, and adjacent relationships with other parts, a unit that detects part type classification and a feature shape from information of the 3D CAD model, a unit that represents an assembly graph from adjacent relationship information between the parts, a unit that generates a disassembly direction and a disassembly order based on the assembly graph and performs inverse conversion to derive an assembly order and an assembly direction, a unit that calculates a part deduction point by multiplying a basic deduction point for each part attachment action by a correction coefficient and calculates an index representing a quality of ease of attachment of the part by subtracting a sum of deduction points for each part from a reference point, a unit that evaluates operability by generating a plurality of virtual operator positions, postures, and viewpoints according to the assembly order, and an overall evaluation unit that calculates an evaluation value of the index of ease of attachment of the part and an overall evaluation value of assembly operability and outputs results”. PTL 2 discloses a technique for generating an operation procedure with a shortest series of operation times based on an assembly operation time of each part and a priority relationship, such as the need to attach a certain part before another part.

### CITATION LIST

### Patent Literature

PTL 1: JP2014-182557A
PTL 2: JP5833998B

### SUMMARY OF INVENTION

### Technical Problem

In the techniques disclosed in PTL 1 and PTL 2, in a maintenance operation for a purpose of keeping an inside of a device clean, there is no consideration for a possibility that the inside of the device may be re-contaminated by a foreign matter adhering in an assembly process after disassembly and cleaning and a disassembly and cleaning operation may have to be performed again. In general, an operator or a tool performing assembly generates debris such as dust, sebum, or sweat. In addition, a part itself may generate dust such as cutting chips generated when a screw is attached or detached. The above debris and cutting chips are collectively referred to as a foreign matter, and a substance that generates the foreign matter is referred to as a foreign matter source in the invention. In the maintenance operation, the inside of the device may be re-contaminated by, for example, an action of gripping an in-device part with a hand, an action of passing the foreign matter source above an exposed in-device part, or an action of gripping the in-device part after touching the foreign matter source with a hand. These re-contamination processes are probabilistic. In the maintenance operation for the purpose of keeping the inside of the device clean, as accumulation of probabilistic re-contamination caused by each action, when a certain amount or more of foreign matter is accumulated in the device and the device is determined to fail in a foreign matter test, the disassembly and cleaning operation is performed again, and a stop time of the device is extended.

The invention has been made in view of such a problem, and an object of the invention is to provide a unit that appropriately selects an assembly order of parts and an operation action in order to prevent re-disassembly and re-cleaning caused by a foreign matter generated during an assembly operation of device maintenance adhering to an inside of a device.

### Solution to Problem

In order to solve the above problems, one representative evaluation system of an operation of the invention is an evaluation system of an operation including a plurality of steps on an object having a surface for which a predetermined level of cleanliness is required, in which the evaluation includes calculating a total sum of a sum of a plurality of operation times required for the respective plurality of steps, and a product of the sum of the operation times and a coefficient obtained in advance indicating a degree of an occurrence of a defect due to a foreign matter deteriorating the level of cleanliness of the surface in the plurality of steps after the operation based on an evaluation result on a degree of diffusion or adhesion of the foreign matter, and the calculated result is displayed or notified on a display.

### Advantageous Effects of Invention

According to the invention, in a maintenance operation for a purpose of keeping an inside of a device clean, it is possible to improve an equipment operation rate by reducing a frequency of occurrence of re-disassembly and re-cleaning due to a foreign matter adhering during assembly and shortening a maintenance time.

Problems, configurations, and effects other than those described above will be made clear by the following description of an embodiment for carrying out the invention.

### BRIEF DESCRIPTION OF DRAWINGS

FIG. 1 is a configuration diagram of an operation procedure generation device in a first embodiment of the invention.

FIG. 2 is a sectional view schematically showing a configuration of a reaction container portion in the first embodiment of the invention.

FIG. 3 is a diagram showing an example of a data format of operation definition information.

FIG. 4 is a diagram showing an example of a data format of foreign matter source information.

FIG. 5 is a diagram showing an example of a data format of calculation result information.

FIG. 6 is a diagram showing an example of a flowchart representing processing in a processing unit.

FIG. 7 is a directed graph showing candidates for an assembly order of the reaction container portion in the first embodiment of the invention.

FIG. 8 is a tree diagram showing candidates for the assembly order of the reaction container portion in the first embodiment of the invention.

FIG. 9 is a view showing an assembly action example of a cylindrical part in the first embodiment of the invention.

FIG. 10 is a view showing an assembly action example of the cylindrical part in the first embodiment of the invention.

FIG. 11 is a directed graph showing a combination of parts and actions for candidates for an assembly procedure of the reaction container portion in the first embodiment of the invention.

FIG. 12 is a diagram showing an example of a flowchart showing processing in a foreign matter defect coefficient unit.

FIG. 13 is a diagram showing an example of a flowchart showing processing in a foreign matter diffusion unit.

FIG. 14 is a diagram showing a GUI example in the first embodiment of the invention.

### DESCRIPTION OF EMBODIMENTS

Hereinafter, an embodiment will be described with reference to the drawings. In the drawings for showing the embodiment, the same parts are denoted by the same reference signs in principle, and the repeated description thereof is omitted.

### First Embodiment

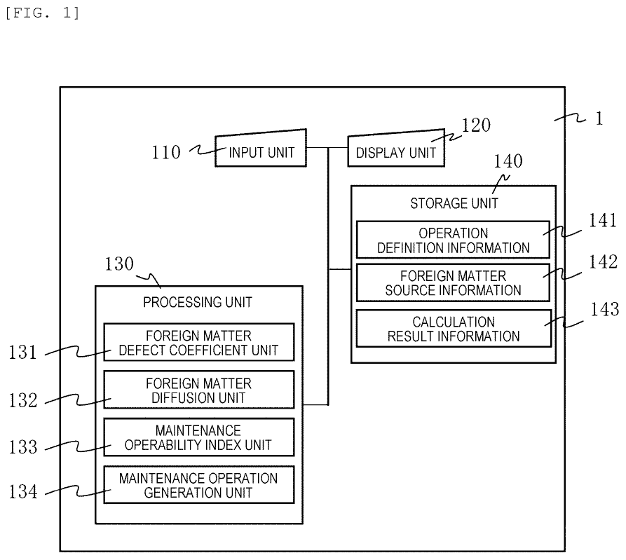

A first embodiment of the invention will be described with reference to FIGS. 1 to 14.

### [Device Configuration]

FIG. 1 is a configuration diagram of an operation procedure generation device 1 in the first embodiment of the invention. The operation procedure generation device 1 includes an input unit 110 that inputs and operates setting information necessary for calculation, a display unit 120 that displays a result, a processing unit 130 that executes operation procedure generation processing, and a storage unit 140 that stores a processing result. In the present disclosure, a case where an operation procedure of a maintenance operation is generated will be described. The input unit 110 includes an input device such as a keyboard, a mouse, a touch panel, or a voice recognition device, the display unit 120 includes a display device, for example, a display, or a projection device such as a projector, or a printer that prints a calculation result, and the processing unit 130 includes a calculation device such as a central processing unit (CPU), a read-only memory (ROM), or a random access memory (RAM) to execute calculation. The storage unit 140 is a storage device such as a hard disk device or a cloud storage having a function of communicating with the operation procedure generation device 1. The input device, the display device, the calculation device, and the storage device described above do not need to be dedicated to the operation procedure generation device 1, and may be devices generally used in a personal computer, a smartphone, or the like.

The processing unit 130 includes a foreign matter defect coefficient unit 131 that calculates a foreign matter defect coefficient 1431 which is an index indicating a risk of failing in a foreign matter test performed after device assembly, a foreign matter diffusion unit 132 that calculates an influence of a foreign matter diffusing between parts, a hand of an operator, and the like, a maintenance operability index unit 133 that evaluates maintenance operability based on an operation time in a series of maintenance operations, the foreign matter defect coefficient 1431, and the like, and a maintenance operation generation unit 134 that selects a maintenance operation that maximizes or minimizes a maintenance operability index. The foreign matter defect coefficient 1431 will be described later.

Although a configuration of the operation procedure generation device 1 has been described, the present disclosure is not limited to the case of the device. Functions of the operation procedure generation device 1 may also be realized by a system in which separate devices are combined. For example, the functions of the operation procedure generation device 1 may also be realized by an evaluation system of an operation including a plurality of steps on an object (reaction container portion 200) having a surface (cleaning surface 207) for which a predetermined level of cleanliness is required. Here, the evaluation includes calculating a total sum of a sum (total operation time 1435) of a plurality of operation times 1413 required for the respective plurality of steps, and a product of the sum (total operation time 1435) of the operation times and a coefficient (total foreign matter defect coefficient 1434) obtained in advance indicating a degree of an occurrence of a defect due to a foreign matter deteriorating the level of cleanliness of the surface in the plurality of steps after the operation based on an evaluation result on a degree of diffusion or adhesion of the foreign matter, and the calculated result is displayed or notified on a display (display unit 120). The cleaning surface 207, the reaction container portion 200, the total foreign matter defect coefficient 1434, and the total operation time 1435 will be described later.

### [Description of Maintenance Target Equipment]

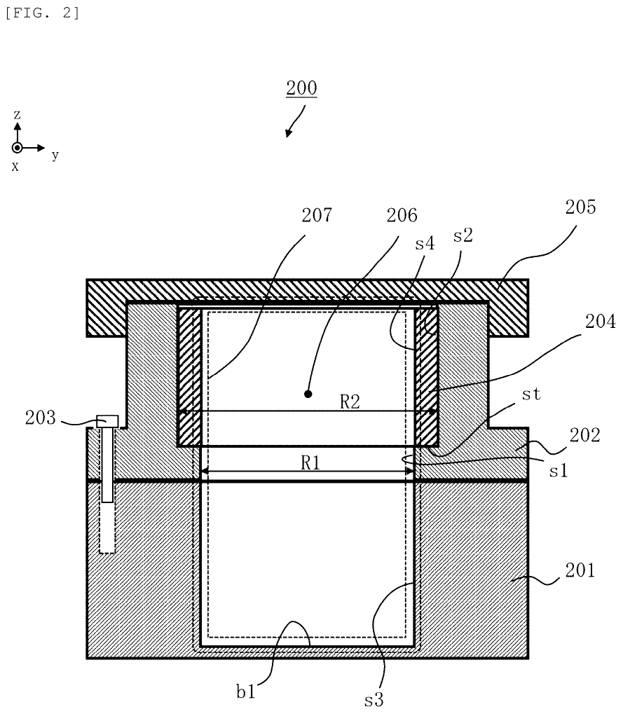

FIG. 2 is a sectional view schematically showing a configuration of the reaction container portion 200 in the first embodiment of the invention. In the present disclosure, the maintenance operation is to construct the object by assembling or attaching a plurality of parts, at least one of which includes the surface (cleaning surface 207) for which the predetermined level of cleanliness is required, or to disassemble the object into at least one of the plurality of parts. Although a case of the reaction container portion 200 will be described as an example of equipment that is a target of the maintenance operation, the present disclosure may also be applied to equipment other than the reaction container portion 200.

The reaction container portion 200 includes a lower container 201, an upper container 202, a screw 203, a cylindrical part 204, and a lid 205, and a cylindrical internal space 206 extending in a z-axis direction is formed inside the reaction container portion 200 by the lower container 201 and the upper container 202. The upper container 202 is attached to the lower container 201, and the upper container 202 and the lower container 201 are sealed by fastening the screw 203 disposed in screw holes respectively provided in the lower container 201 and the upper container 202.

Describing each part, the upper container 202 has a cylindrical through hole therein. The through hole has a shape obtained by combining a cylindrical portion having a diameter R1 formed by a side surface s1 and a cylindrical portion having a diameter R2 formed by a wall surface s2, and a step st is formed at a boundary between the two cylindrical portions. The step st has a ring shape when viewed from a z-axis positive direction, and the cylindrical part 204 is disposed on the step st. The cylindrical part 204 is a member having a ring shape, and is fitted along the wall surface s2 of the upper container 202. The lid 205 is placed on an opening of the upper container 202 in the z-axis positive direction so as to separate the upper container 202 from the outside.

The lower container 201 has a cylindrical recess formed by a bottom surface bl and a side surface s3. A diameter of the recess is equal to the diameter R1 of the cylindrical portion of the upper container 202.

For example, when the reaction container portion 200 is used in a semiconductor manufacturing device, reaction processing related to semiconductor manufacturing is performed in the internal space 206, but a reaction product may adhere to an inner wall of the reaction container portion 200. Therefore, an operation is necessary to disassemble and clean the reaction container portion 200. In addition, when the container is assembled after disassembly and cleaning, it is necessary to avoid re-adhesion of a foreign matter, and in particular, a bottom surface (surface on a z-axis negative direction side) of the lid 205 constituting a wall surface of the internal space 206, the bottom surface bl of the lower container 201, the side surface s1, and an inner wall surface s4 of the cylindrical part 204 are portions where foreign matter adhesion is to be avoided. Hereinafter, the lid 205, the side surface s3, the wall surface s4, and the bottom surface bl are collectively referred to as the cleaning surface 207. When the foreign matter adheres to the cleaning surface 207, the foreign matter test after assembly fails and a disassembly and cleaning operation is performed again, which increases a total maintenance operation time.

The above description shows a main configuration of the reaction container portion 200, and the present disclosure is not limited to this configuration. In addition to the cylindrical part 204, a part necessary for a manufacturing step, such as a sensor that measures a temperature or the like in the internal space 206, may be disposed in the reaction container portion 200. In addition, a gas pipe or an exhaust pipe for introducing a gas from the outside, a part for transporting a member from another container portion, or the like may be connected to the reaction container portion 200.

### [Description of Information Stored in Storage Unit]

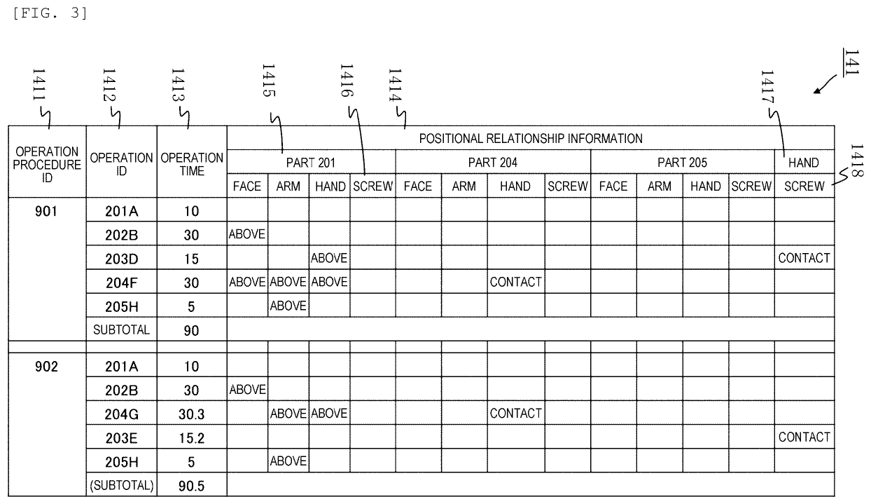

Hereinafter, examples of data formats of operation definition information 141, foreign matter source information 142, and calculation result information 143 stored in the storage unit 140 will be described with reference to FIGS. 3 to 5. In the evaluation system of the operation, for each of the plurality of steps, the coefficient (total foreign matter defect coefficient 1434) indicating the degree of the occurrence of the defect due to the foreign matter after the operation is calculated using information (positional relationship information 1414) obtained in advance on a relative position of the part (cleaning part 1415) and a foreign matter source 1416 including at least one portion of a body of an operator. Hereinafter, details will be described.

FIG. 3 is a diagram showing the example of the data format of the operation definition information 141. The operation definition information 141 lists an operation procedure ID 1411 assigned to the operation procedure and an operation ID 1412 arranged in an operation order. Further, for each operation ID 1412, the operation time 1413 and the positional relationship information 1414 indicating the relative positional relationship of the foreign matter source 1416 and the cleaning part 1415 which is a part for which the adhered foreign matter is evaluated are stored. In the present embodiment, the cleaning part 1415 for which a foreign matter adhesion index is evaluated is the lid 205 (corresponding to a “part 205” in FIG. 3) constituting the cleaning surface 207, the lower container 201 (corresponding to a “part 201”), and the cylindrical part 204 (corresponding to a “part 204”).

The positional relationship information 1414 is information defined as one of three types, that is, “contact”, “above”, and “ignoring (displayed by blank)”. The “contact” indicates that the cleaning surface 207 of the cleaning part 1415 and the foreign matter source 1416 are in contact with each other, the “above” indicates that the foreign matter source 1416 is present above the cleaning surface 207 in a non-contact state, that is, in a direction opposite to gravity, and the “ignoring” indicates that the cleaning surface 207 is not affected by the foreign matter source 1416.

Examples of the representative foreign matter source 1416 include, but are not limited to, a “face”, an “arm”, and a “hand” of an operator, and a “screw” to which cutting chips that may be generated when the screw is removed or tightened adhere. In addition, the cleaning part 1415 may include, in addition to the part constituting the wall surface of the internal space 206 described above, a hand of an operator or the like that needs to avoid adhesion of a foreign matter because of coming into contact with the cleaning surface 207. The operation definition information 141 is used to calculate the foreign matter defect coefficient 1431 and the maintenance operability index to be described later.

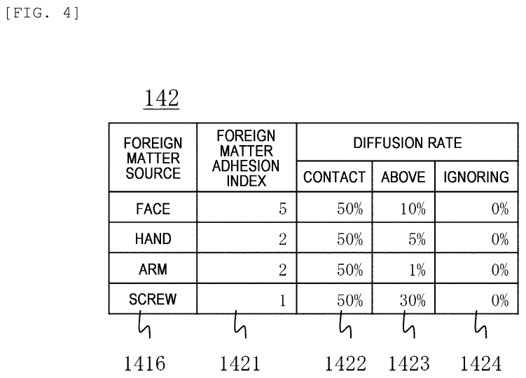

FIG. 4 is a diagram showing the example of the data format of the foreign matter source information 142. The foreign matter source information 142 stores, for each foreign matter source 1416, a foreign matter adhesion index 1421 indicating an amount of foreign matter, a contact foreign matter diffusion rate 1422 indicating foreign matter diffusion due to contact, and an above foreign matter diffusion rate 1423 indicating foreign matter diffusion due to a foreign matter falling from the above. In general, when the foreign matter source 1416 and the part come into contact with each other, a part of a foreign matter on a surface of the foreign matter source moves to the part in contact with the foreign matter source, and a foreign matter peeled off from the foreign matter source 1416 above an attachment destination part may fall onto the attachment destination part or another foreign matter source due to gravity. The foreign matter adhesion index 1421 is defined based on such a physical phenomenon, and is used to calculate the diffusion of the foreign matter or likelihood of an occurrence of a foreign matter defect according to the relative positional relationship such as “contact” or “above” in foreign matter diffusion calculation processing or foreign matter defect coefficient calculation processing to be described later. When the relative positional relationship between the foreign matter source and the cleaning part 1415 is “ignoring”, foreign matter adhesion does not occur, and thus 0% is input as an ignoring foreign matter diffusion rate 1424.

The foreign matter source information 142 is given as input information, and the given foreign matter adhesion index 1421 is an initial value, and the foreign matter adhesion index changes as the foreign matter diffuses in each operation. For example, a procedure can be evaluated based on an increase in the foreign matter adhesion index 1421 due to foreign matter diffusion between foreign matter sources, such as an increase in the foreign matter adhesion index 1421 for a hand by touching a screw and then touching a part with the hand with cutting chips.

The foreign matter adhesion index 1421 relatively represents an influence of failing in the foreign matter test, and can be determined by, for example, experimentally obtaining the number of foreign matters generated and adhered during operation, a foreign matter defect rate which is a fail rate of the foreign matter test, and the like. In addition, the contact foreign matter diffusion rate 1422 and the above foreign matter diffusion rate 1423 can also be determined experimentally, for example. In the embodiment described below, the foreign matter adhesion index 1421 is described as the foreign matter defect rate (%). The definition of the foreign matter adhesion index 1421 is not limited thereto, and for example, an index such as the number of foreign matters adhering to a part, the number of foreign matters adhering to a foreign matter source, or an area of a portion to which foreign matters adhere may be used. In addition, for example, a relative value indicating the likelihood of the occurrence of the foreign matter defect rate such as a ratio based on the number of foreign matters or an area of a certain foreign matter source may be used. In particular, in a case of using the relative value, for example, at the time of handling a part that is 1.5 times larger, the number of foreign matters will also be 1.5 times larger, which saves the time and effort of experimentally investigating.

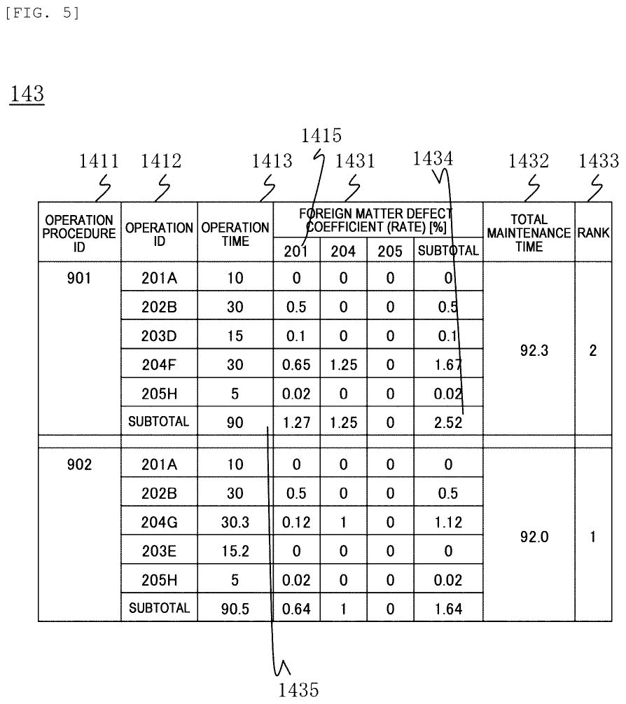

FIG. 5 is a diagram showing the example of the data format of the calculation result information 143. The operation procedure ID 1411, the operation ID 1412, and the operation time 1413 are the same as those included in the operation definition information 141 shown in FIG. 3. The maintenance operability index unit 133 calculates the foreign matter defect coefficient 1431, a total maintenance time 1432, and a rank 1433 for each operation ID 1412, and stores the foreign matter defect coefficient 1431, the total maintenance time 1432, and the rank 1433 in the storage unit 140 (calculation method will be described later). The foreign matter defect coefficient 1431 is an index indicating an influence of a failure in the foreign matter test due to a foreign matter adhering to a certain part in a certain operation, and is described as a defect rate similarly to the foreign matter adhesion index 1421 in FIG. 3 in the present embodiment. A definition of the foreign matter defect coefficient 1431 is not limited thereto, and other values such as the number of foreign matters adhering to a part or an area of a portion to which foreign matters adhere may be used according to a content of the foreign matter adhesion index 1421. In addition, in the following embodiment, the total maintenance time 1432 is treated as a maintenance operability index, and an index such as a foreign matter defect rate in a series of operations, the total number of foreign matters adhering to the equipment, a total area where foreign matters adhere, a total of foreign matter defect coefficients, or a total operation time may be used as the maintenance operability index.

The foreign matter defect coefficient 1431 is calculated for each cleaning part 1415, and then a subtotal is calculated to obtain the foreign matter defect coefficient 1431 for each operation ID 1412. The total maintenance time 1432 is calculated for each operation procedure ID based on the operation time 1413 and the foreign matter defect coefficient 1431, and is stored in the storage unit 140 (calculation method will be described later) The rank 1433 is a rank when the total maintenance time 1432 as the maintenance operability index in the present embodiment is arranged in ascending order.

### [Description of Calculation Procedure]

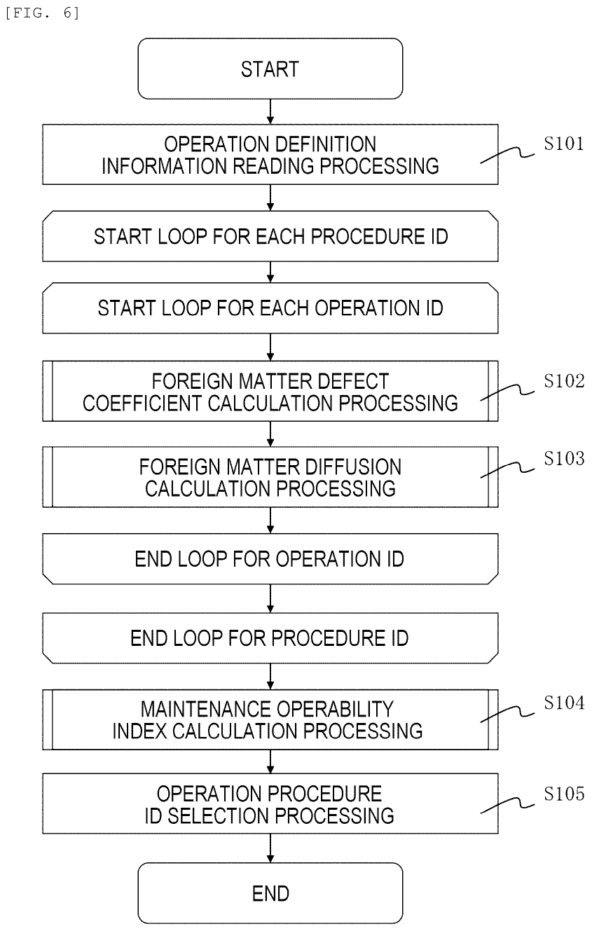

FIG. 6 is a diagram showing an example of a flowchart representing processing in the processing unit 130. For each of the plurality of steps, the coefficient (total foreign matter defect coefficient 1434) indicating a degree of an occurrence of a defect due to the foreign matter after the operation is calculated based on a product of the degree of adhesion of the foreign matter of the foreign matter source to the part targeted in the step and the degree of diffusion of the foreign matter in the step.

Specifically, the processing unit 130 performs operation definition information reading processing (S101). Subsequently, the processing unit 130 repeatedly performs the foreign matter defect coefficient calculation processing (S102) and the foreign matter diffusion calculation processing (S103) for each operation procedure ID 1411 and each operation ID 1412. Subsequently, the processing unit 130 performs maintenance operability index calculation processing (S104) and operation procedure ID selection processing (S105).

According to the flowchart of FIG. 6, a plurality of candidates of the operation procedure (hereinafter, also referred to as “operation procedure candidates”) are comprehensively calculated, and then a total maintenance time in each operation procedure is compared, and for example, an operation procedure having a shortest total maintenance time can be output. As other processing, for example, a procedure may be generated by sequentially selecting operations with a minimum total maintenance time, and a general method such as a greedy algorithm, an A * (A star) algorithm, or a Dijkstra's algorithm may be used. Hereinafter, an example of each processing content described in the entire processing flow will be described.

### (Operation Definition Information Reading Processing)

The operation definition information reading processing S101 will be described. In the operation definition information reading processing S101, the operation procedure candidates obtained by combining an assembly order and an assembly action of the plurality of parts are read from the operation definition information 141. Here, information indicating the operation procedure candidates described in the operation definition information 141 will be described with reference to FIGS. 7 to 11.

### (Generating Method of Operation Procedure Candidates)

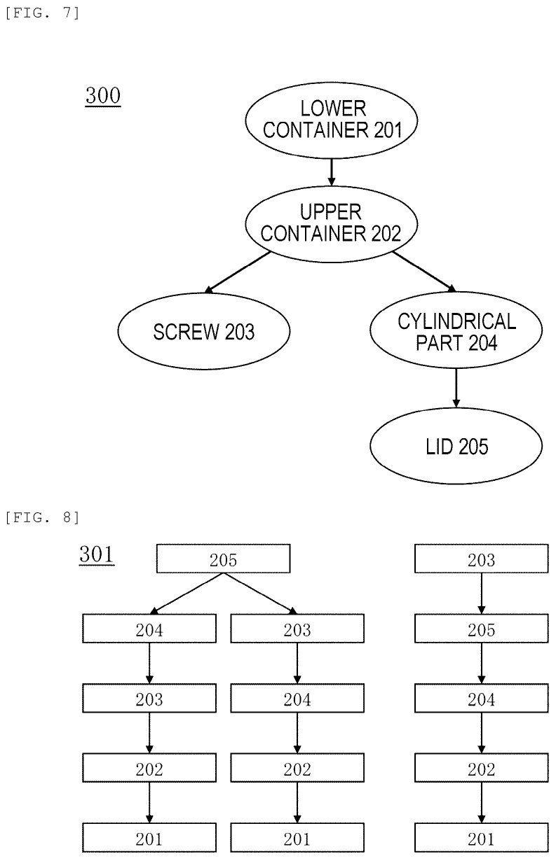

A method of generating the assembly order (hereinafter, also referred to as an “assembly sequence”) using the reaction container portion 200 as an example will be described with reference to FIGS. 7 and 8. FIG. 7 is a directed graph showing candidates for an assembly order of the reaction container portion in the first embodiment of the invention. FIG. 8 is a tree diagram showing candidates for the assembly order of the reaction container portion in the first embodiment of the invention.

In a directed graph 300 shown in FIG. 7, vertices shown as ellipses represent parts, and arrows connecting the vertices represent precedence relationships of assembly. For example, the arrow between the lid 205 and the cylindrical part 204 indicates that the cylindrical part 204 connected to an arrow base is assembled and then the lid 205 connected to an arrow tip is attached. In the directed graph 300, a part corresponding to a vertex at which all connected arrows are arrow tips is a part to be attached last, and the lid 205 and the screw 203 correspond thereto. In addition, a part corresponding to a vertex at which all connected arrows are arrow bases is a part to be attached first, and the lower container 201 corresponds thereto.

As a method of generating the assembly order based on the directed graph 300, there is a method using a tree diagram. First, candidates for the part to be attached last are arranged in a first hierarchical level of the tree diagram. Next, one part is selected from the arranged parts, and parts that can be disassembled when the part is removed are arranged in a second hierarchical level. The above processing is recursively performed until there is no part candidate to be removed. In a case of the directed graph 300, the screw 203 and the lid 205 are parts that can be disassembled in an initial state. When the lid 205 is removed, the screw 203 and the cylindrical part 204 are to be disassembled next, and thus the screw 203 and the cylindrical part 204 are arranged in the second hierarchical level, and a branch extending from the lid 205 to the screw 203 and a branch extending from the lid 205 to the cylindrical part 204 are added. A tree diagram 301 obtained by repeating the above is shown in FIG. 8. A disassembly order is obtained by tracing FIG. 8 from the first hierarchical level, and the following three assembly orders are obtained by reversing this order.

(Assembly sequence 1) The upper container 202 is attached to an upper portion where the lower container 201 is installed, the cylindrical part 204 is inserted into the upper container 202, the lid 205 is closed, and finally the screw 203 is fastened.

(Assembly sequence 2) The upper container 202 is attached to an upper portion where the lower container 201 is installed, the cylindrical part 204 is inserted into the upper container 202, the screw 203 is fastened, and finally the lid 205 is closed.

(Assembly sequence 3) The upper container 202 is attached to an upper portion where the lower container 201 is installed and fastened with the screw 203, the cylindrical part 204 is inserted into the upper container 202, and finally the lid 205 is closed.

Also in a case where there is a sub-assembly including a plurality of parts, an assembly sequence can be generated in the same manner. For example, a procedure can be generated by treating a sub-assembly as one part and generating an assembly sequence, then similarly generating an assembly sequence for each sub-assembly, and inserting an operation for assembling the sub-assembly before assembling each sub-assembly.

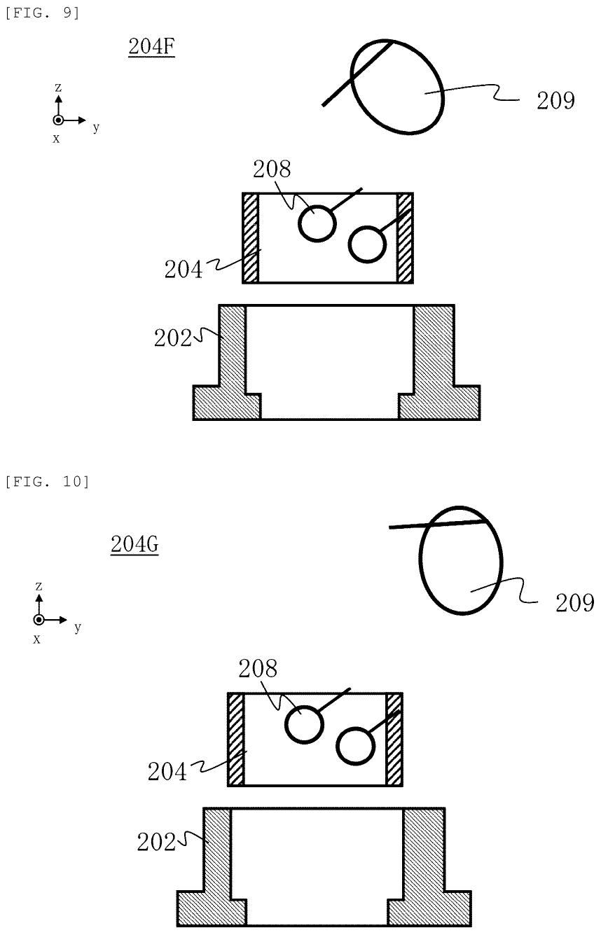

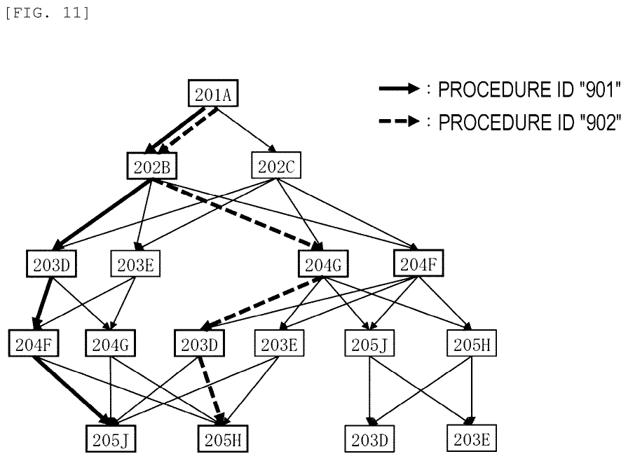

Next, an example in which a plurality of actions exist in an assembly operation will be described with reference to FIGS. 9 and 10. FIGS. 9 and 10 are views showing assembly action examples of the cylindrical part 204 in the first embodiment of the invention.

FIG. 9 is an example showing an action F of attaching the cylindrical part. Specifically, as the action F, an operator places a face 209 above the upper container 202 such that the operator can look into a through hole of the upper container 202, and inserts the cylindrical part 204 gripped by a hand 208 into the upper container 202 while checking an insertion state of the cylindrical part 204. An action A corresponds to an operation ID “204F” in FIG. 3.

FIG. 10 is an example showing an action G of attaching the cylindrical part. An action B is an action of inserting the cylindrical part 204 into the upper container 202 while making sure the face 209 is not above the through hole of the upper container 202, and corresponds to an operation ID “204G” in FIG. 3. As described above, the assembly action of the part can take a plurality of patterns according to a position of the foreign matter source (face or hand) during the action, a movement path of the part, and the like.

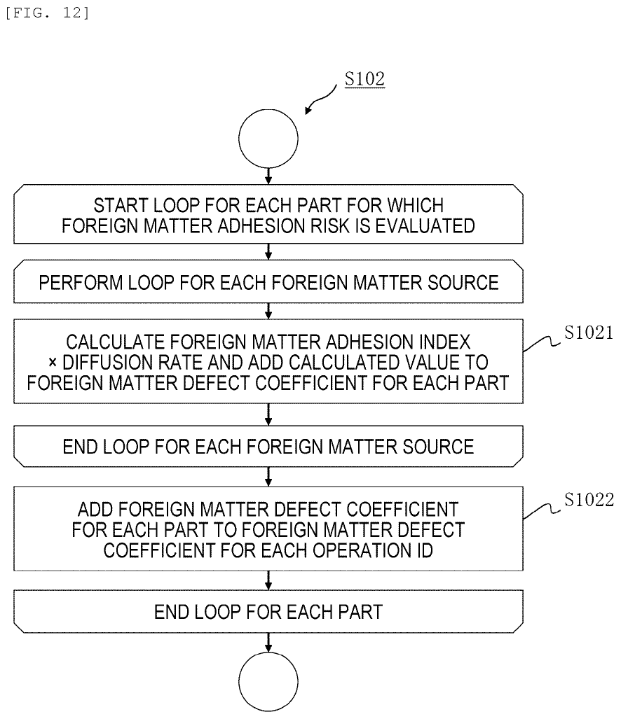

Next, an example of an assembly order in which possible action patterns are combined from (assembly sequence 1) to (assembly sequence 3) described above will be described with reference to FIG. 11. FIG. 11 is a directed graph showing a combination of parts and actions for candidates for an assembly procedure of the reaction container portion in the first embodiment of the invention. In FIG. 11, the action A to an action H are shown as examples, and one or two assembly actions are assigned to one part (the action A is assigned to the part (lower container) 201, the action B and the action C are assigned to a part (upper container) 202, the action D and the action E are assigned to a part (screw) 203, the action F and the action G are assigned to the part (cylindrical part) 204, and the action H and an action J are assigned to the part (lid) 205), and three or more assembly actions may be assigned to one part. In FIG. 11, for example, when the part 201 is assembled by the action A, the action is represented as “201A”, which corresponds to an operation ID in FIG. 3. In addition, a procedure ID “901” (specifically, procedures of 201A, 202B, 203D, 204F, and 205J) of the operation definition information 141 which is the input information shown in FIG. 3 is a data example of an operation order indicated by thick solid arrows in FIG. 11, and a procedure ID “902” (specifically, procedures of 201A, 202B, 204G, 203D, and 205H) is a data example of an operation order indicated by thick dotted arrows in FIG. 11.

Regarding the operation definition information 141, the directed graph 300 showing a preceding order of assembly including a constraint condition between parts is derived from a 3D model of the device by using a general constraint condition extraction technique, and an possible operation posture is calculated from the 3D model in which a human body model is additionally disposed by using a general line simulation technique, and thus the operation procedure ID 1411 and the operation ID 1412 may be comprehensively generated or the operation time 1413 may be calculated by the same technique. Further, a general ray tracing technique can be used to perform ray tracing in the direction opposite to gravity from the cleaning surface 207 and to check whether a ray intersects with a face, an arm, a hand, or the like which is the foreign matter source, so as to calculate the relative positional relationship such as “contact” or “above”, and adopt the relative positional relationship as the positional relationship information 1414.

### (Foreign Matter Defect Coefficient Calculation Processing)

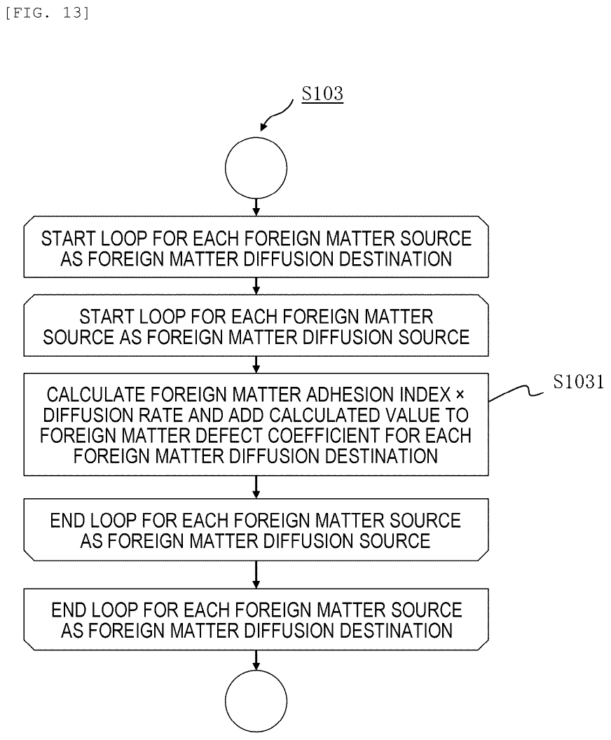

Next, the foreign matter defect coefficient calculation processing S102 of FIG. 6 will be described in detail with reference to FIG. 12. FIG. 12 is a diagram showing an example of a flowchart showing processing in the foreign matter defect coefficient unit 131. In the foreign matter defect coefficient calculation processing S102, the foreign matter defect coefficient 1431 is calculated for each operation ID 1412 based on the positional relationship information 1414 of FIG. 3. For example, in the operation ID “204G” shown in FIG. 10, since the arm or the hand which is the foreign matter source is located above with respect to the part “201”, a foreign matter adhesion index (foreign matter defect rate) of the hand of “2%” and an above foreign matter diffusion rate of “5%” are multiplied based on the foreign matter source information 142 shown in FIG. 4 to obtain a foreign matter defect coefficient of “0.1%”. Similarly, the calculation is performed for each foreign matter source, and “0.02%” is obtained as a foreign matter defect coefficient due to the arm. Further, these coefficients are added to obtain a foreign matter defect coefficient for the part “201” of “0.12%” (step S1021). Similarly, a foreign matter defect coefficient is calculated for each part, and a foreign matter defect coefficient of the part “204” of “1%” is obtained. The part “205” has a foreign matter defect coefficient of “0%” because all pieces of positional relationship information 1414 are “ignoring (indicated by blank)”. As described above, a subtotal of the foreign matter defect coefficient 1431 for the operation ID “204G” is calculated as “1.12%” and stored in a subtotal field of the foreign matter defect coefficient 1431 (step S1022). A calculation order of the foreign matter source, the operation ID 1412, and the part is not limited to the above-described case, and the order is not limited as long as the calculation can be performed comprehensively.

### (Foreign Matter Diffusion Calculation Processing)

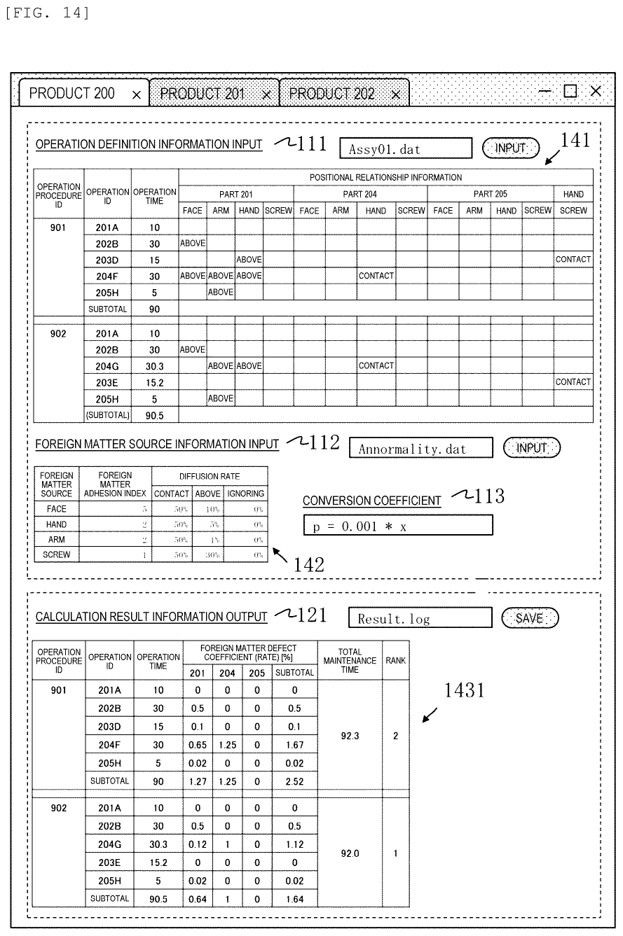

Next, the foreign matter diffusion calculation processing S103 of FIG. 6 will be described with reference to FIG. 13. FIG. 13 is a diagram showing an example of a flowchart showing processing in the foreign matter diffusion unit. In the foreign matter diffusion calculation processing S103, the foreign matter diffusion that may occur between the foreign matter sources is considered based on the positional relationship information 1414.

For example, in the operation ID “203D” that is a screw fastening operation, the hand which is the foreign matter source to be a foreign matter diffusion destination 1417 and the screw which is a foreign matter source to be a foreign matter diffusion source 1418 are extracted from the positional relationship information 1414. In a case of “contact” by gripping the screw which is the foreign matter diffusion source with the hand, a foreign matter adhesion coefficient of the screw of “1%” and a contact diffusion rate of “50%” are multiplied based on the foreign matter source information 142 to obtain “0.5%” as a foreign matter diffusion amount. By adding the foreign matter diffusion amount to the foreign matter adhesion index of the hand of “2%” (step S1031), a new foreign matter adhesion index of “2.5%” is calculated, and a value of the foreign matter adhesion index 1421 is updated. According to the operation order of the operation ID 1412, the processing is sequentially performed for each foreign matter source as a foreign matter diffusion destination and for each foreign matter source as a foreign matter diffusion source, so that an influence of the foreign matter diffusion can be calculated.

### (Maintenance Operability Index Calculation Processing)

Next, the maintenance operability index calculation processing S104 of FIG. 6 will be described in detail. The coefficient (total foreign matter defect coefficient 1434) indicating the degree of the occurrence of the defect due to the foreign matter after the operation is calculated as a sum (total maintenance time 1432) of the product of the degree of adhesion of the foreign matter (foreign matter adhesion index 1421) and the degree of diffusion (contact foreign matter diffusion rate 1422 and above foreign matter diffusion rate 1423) for each of the plurality of steps. In the present embodiment, a case where the total maintenance time 1432 is used as the maintenance operability index will be described, but requirements other than the total maintenance time 1432 are also adopted as evaluation targets. Specifically, the foreign matter defect coefficient 1431 for each operation ID stored in the calculation result information 143 is added for each operation procedure ID 1411.

For example, in the operation procedure ID “901”, a total foreign matter defect coefficient of “2.52%” is calculated by adding a subtotal of the foreign matter defect coefficients of the operation ID “201A” to “205H”, and is stored in the total foreign matter defect coefficient 1434. As the total operation time, the operation times 1413 of the operation ID “201A” to “205H” are added and stored in the total operation time 1435.

The total maintenance time 1432 is calculated using, for example, Equation (1). A total foreign matter defect rate is a probability that a foreign matter defect occurs in at least one operation in a series of operations, and is equal to the total foreign matter defect coefficient 1434.

Total maintenance time=total operation time+total operation time×total foreign matter defect rate (1)

As in the present embodiment, when a foreign matter adhesion index is defined as an occurrence rate of a foreign matter defect due to an action in which a foreign matter source comes into a predetermined positional relationship with respect to a part, the total foreign matter defect rate is equal to the total foreign matter defect coefficient 1434 on an assumption that each defect rate is sufficiently small.

Unlike the present embodiment, when a foreign matter adhesion coefficient is defined as, for example, the total number of foreign matters adhered to the equipment in a series of operations, a total surface area of a portion to which the foreign matter adheres, or the like, and the total maintenance time is set as the maintenance operability index, the total foreign matter defect rate can be determined based on the total foreign matter defect coefficient 1434 by a unit of experimentally obtaining a correlation coefficient representing a correlation between the total foreign matter defect coefficient 1434 and the total foreign matter defect rate, for example. In other words, the coefficient (total foreign matter defect coefficient 1434) indicating the degree of the occurrence of the defect due to the foreign matter after the operation is calculated using a coefficient (correlation coefficient) obtained in advance indicating a correlation between a value of the sum of the product of the degree of adhesion of the foreign matter (foreign matter adhesion index 1421) and the degree of diffusion (contact foreign matter diffusion rate 1422 and above foreign matter diffusion rate 1423) for each of the plurality of steps and the degree of the occurrence of the defect due to the foreign matter after the operation.

### (Operation Procedure ID Selection Processing)

Next, the operation procedure ID selection processing S105 will be described in detail. In S105, an optimized operation procedure ID is selected. Specifically, among a plurality of operations for the object which achieve the same result, one operation in which the total maintenance time 1432 is the smallest is displayed or notified. In the present embodiment, the total maintenance time 1432 is used as the maintenance operability index, but the invention is not limited thereto. The total maintenance time 1432 for each operation procedure ID stored in the calculation result information 143 is numbered in ascending order and stored in the rank 1433. Of these, a procedure ID having a first rank is output. Here, the ranking is performed in ascending order of the total maintenance time based on Equation (1), but other evaluation methods can also be used, such as a method of ranking only based on the total operation time and a method of ranking in ascending order of the total foreign matter defect coefficient.

### [Description of GUI]

Next, an input/output GUI example of the above operation procedure generation device 1 will be described with reference to FIG. 14. FIG. 14 is a diagram showing the GUI example in the first embodiment of the invention. FIG. 14 shows the GUI example in a case of the reaction container portion 200 (product 200). The operation definition information 141 is input from an operation definition information input unit 111, the foreign matter source information 142 is input from a foreign matter source information input unit 112, and the calculation result information 143 is displayed via a calculation result information display unit 121. For example, a content of “Assy01.dat” set in the operation definition information input unit 111 is displayed in the operation definition information 141, and a content of “Annormality.dat” set in the foreign matter source information input unit 112 is displayed in the foreign matter source information 142. A conversion coefficient input unit 113 is an input unit that sets the correlation coefficient representing the correlation between the total foreign matter defect coefficient 1434 and the total foreign matter defect rate.

A user creates a procedure manual using a first operation displayed in the calculation result information 143 as a master operation. The procedure manual is output as “Result.log”.

### Function and Effects

As described above, according to the present disclosure, in a maintenance operation for a purpose of keeping an inside of a device clean, it is possible to improve an equipment operation rate by reducing a frequency of occurrence of re-disassembly and re-cleaning due to a foreign matter adhering during assembly and shortening a maintenance time.

The invention is not limited to the embodiment described above, and includes various modifications. For example, the embodiment described above has been described in detail to facilitate understanding of the invention, and the invention is not necessarily limited to those including all configurations described above. A part of a configuration of one embodiment can be replaced with or added to a configuration of another embodiment. In addition, it is possible to add, delete, or replace a part of configurations of each embodiment with other configurations.

For example, a case of a maintenance operation of assembling the reaction container portion 200 has been described, but the present disclosure can also be applied to an operation of disassembling as in a case of disassembling the reaction container portion 200 and extracting the upper container 202.

A part or all of configurations, function portions, processing units, and the like described above may be implemented as hardware by, for example, designing with an integrated circuit. In addition, a part or all of the configurations, functions, and the like described above may be implemented, for example, by a person executing the configurations, functions, and the like, or by a processor interpreting and executing a program for implementing each function. Information such as a program, a table, and a file for implementing each function can be stored in a recording device such as a memory, a hard disk, and a solid state drive (SSD), or in a recording medium such as an IC card, an SD card, and a DVD, or in a distributed recording device such as a cloud storage service.

Aspects that may be contents of the invention will be described below, but the invention is not limited thereto.

### (Aspect 1)

An evaluation system of an operation including a plurality of steps on an object having a surface for which a predetermined level of cleanliness is required, in which
the evaluation includes calculating a total sum of a sum of a plurality of operation times required for the respective plurality of steps, and a product of the sum of the operation times and a coefficient obtained in advance indicating a degree of an occurrence of a defect due to a foreign matter deteriorating the level of cleanliness of the surface in the plurality of steps after the operation based on an evaluation result on a degree of diffusion or adhesion of the foreign matter, and
the calculated result is displayed or notified on a display.

### (Aspect 2)

The evaluation system of the operation according to aspect 1, in which
the operation is to construct the object by assembling or attaching a plurality of parts, at least one of which includes the surface for which the predetermined level of cleanliness is required, or to disassemble the object into at least one of the plurality of parts.

### (Aspect 3)

The evaluation system of the operation according to aspect 1 or 2, in which
the evaluation includes calculating, for each of the plurality of steps, the coefficient indicating the degree of the occurrence of the defect due to the foreign matter after the operation, using information obtained in advance on a relative position of the part and a foreign matter source including at least one portion of a body of an operator.

### (Aspect 4)

The evaluation system of the operation according to any one of aspects 1 to 3, in which
the evaluation includes calculating, for each of the plurality of steps, the coefficient indicating the degree of the occurrence of the defect due to the foreign matter after the operation, based on a product of the degree of adhesion of the foreign matter from the foreign matter source to the part targeted in the step and the degree of diffusion of the foreign matter in the step.

### (Aspect 5)

The evaluation system of the operation according to any one of aspects 1 to 4, in which
the evaluation includes calculating a sum of the product of the degree of adhesion and the degree of diffusion of the foreign matter for each of the plurality of steps, as the coefficient indicating the degree of the occurrence of the defect due to the foreign matter after the operation.

### (Aspect 6)

The evaluation system of the operation according to any one of aspects 1 to 5, in which
the evaluation includes calculating the coefficient indicating the degree of the occurrence of the defect due to the foreign matter after the operation by using a coefficient obtained in advance indicating a correlation between a value of the sum of the product of the degree of adhesion and the degree of diffusion of the foreign matter for each of the plurality of steps and the degree of the occurrence of the defect due to the foreign matter after the operation.

### (Aspect 7)

The evaluation system of the operation according to any one of aspects 1 to 6, in which
among the plurality of operations for the object which achieve the same result, one operation in which the total sum is the smallest is displayed or notified.

### (Aspect a)

An operation procedure generation device for calculating an operation procedure of an operation of sequentially assembling and/or an operation of disassembling a plurality of parts, the operation procedure generation device including:
a foreign matter defect coefficient unit, a foreign matter diffusion unit, a maintenance operability index unit, and a maintenance operation generation unit, in which
the foreign matter defect coefficient unit is configured to acquire
a plurality of operation procedures of the operation,
a plurality of actions in each operation,
a relative positional relationship between a cleaning part and a foreign matter source in each action, and
a foreign matter adhesion index of each foreign matter source to calculate a foreign matter defect coefficient as a sum of the foreign matter adhesion indexes due to the foreign matter sources for each action and for each part,
the foreign matter diffusion unit is configured to acquire
a relative positional relationship between the cleaning part and the foreign matter source in each of the plurality of actions,
a foreign matter adhesion index of each foreign matter source, and
a foreign matter diffusion rate of each foreign matter source to calculate a change in the foreign matter adhesion index due to foreign matter diffusion for each action, based on a diffusion rate of a foreign matter and the foreign matter adhesion index of each foreign matter source which are associated with the relative positional relationship,
the maintenance operability index unit is configured to acquire at least one of
an operation time for each action, and
a foreign matter defect coefficient for each part and for each action calculated by the foreign matter defect coefficient unit and the foreign matter diffusion unit, so as to calculate a maintenance operability index that is an index for evaluating each of the operation procedures, and
the maintenance operation generation unit is configured to select an operation procedure having a maximum or minimum maintenance operability index calculated by the maintenance operability index unit.

### (Aspect b)

The operation procedure generation device according to aspect a, in which
the relative positional relationship is defined as one of three states, that is, a state in which the part and the foreign matter source are in contact with each other (hereinafter, referred to as contact), a state in which the foreign matter source is present above the part in a non-contact state (hereinafter, referred to as above), and a state in which the part is not affected by the foreign matter source (hereinafter, referred to as ignoring),
the foreign matter diffusion rate is information in which two types are defined, that is, a contact foreign matter diffusion rate due to contact between the part and the foreign matter source and an above foreign matter diffusion rate due to a foreign matter falling from the foreign matter source above, and
the foreign matter diffusion calculation unit is configured to update the foreign matter adhesion index due to the foreign matter diffusion based on the contact foreign matter diffusion rate when the relative positional relationship is contact or based on the above foreign matter diffusion rate when the relative positional relationship is above, and omit update processing when the relative positional relationship is ignoring.

### (Aspect c)

The operation procedure generation device according to aspect a or b, in which
the foreign matter adhesion index is the number of foreign matters generated from the foreign matter source and adhering to the part.

### (Aspect d)

The operation procedure generation device according to any one of aspects a to c, in which
the foreign matter adhesion index is a defect rate of an action in which the foreign matter source and the part have a positional relationship defined in the relative positional relationship.

### (Aspect e)

The operation procedure generation device according to any one of aspects a to d, in which
the foreign matter adhesion index is an area of a part surface portion occupied by a foreign matter generated from the foreign matter source and adhering to the part.

### (Aspect f)

The operation procedure generation device according to any one of aspects a to e, in which
the foreign matter adhesion index is the number of foreign matters adhering to the foreign matter source.

### (Aspect g)

The operation procedure generation device according to any one of aspects a to f, in which
the foreign matter adhesion index is a ratio of any one of indexes, the number of foreign matters generated from the foreign matter source and adhering to the part, the number of foreign matters adhering to the foreign matter source, and an area of a part surface portion occupied by a foreign matter, to a value in a certain foreign matter source.

### (Aspect h)

The operation procedure generation device according to any one of aspects a to g, in which
the maintenance operability index is a total operation time required for the operation according to the operation procedure.

### (Aspect i)

The operation procedure generation device according to any one of aspects a to h, in which
the maintenance operability index is a total foreign matter defect coefficient obtained by adding the foreign matter defect coefficient in the operation according to the operation procedure for all actions and parts.

### (Aspect j)

The operation procedure generation device according to any one of aspects a to i, in which
the maintenance operability index is the total number of foreign matters adhering in the operation according to the operation procedure.

### (Aspect k)

The operation procedure generation device according to any one of aspects a to j, in which
the maintenance operability index is a total area of the parts to which the foreign matter adheres in the operation according to the operation procedure.

### (Aspect 1)

The operation procedure generation device according to any one of aspects a to k, in which
the maintenance operability index is one obtained by adding the total operation time and a value obtained by multiplying the total foreign matter defect coefficient by the total operation time.

### (Aspect m)

The operation procedure generation device according to any one of aspects a to l, in which
the maintenance operability index unit further includes a unit configured to acquire an estimation function for estimating a foreign matter defect rate from the foreign matter defect coefficient, and the maintenance operability evaluation index is a value obtained by adding the total operation time and a value obtained by multiplying, by the total operation time, the foreign matter defect rate estimated from the total foreign matter defect coefficient by the estimation function.

### (Aspect n)

An operation procedure generation method for calculating an operation procedure of an operation of sequentially assembling and/or an operation of disassembling a plurality of parts, the operation procedure generation method including:
acquiring a plurality of operation procedures of the operation,
a plurality of actions in each operation,
a relative positional relationship between a cleaning part and a foreign matter source in each action,
a foreign matter adhesion index of each foreign matter source,
a foreign matter diffusion rate of each foreign matter source, and
an operation time for each action to calculate a change in the foreign matter adhesion index due to foreign matter diffusion for each action, based on a diffusion rate of a foreign matter and the foreign matter adhesion index of each foreign matter source which are associated with the relative positional relationship;
calculating a foreign matter defect coefficient as a sum of the foreign matter adhesion indexes due to the foreign matter sources for each action and for each part;
calculating a maintenance operability index that is an index for evaluating each of the operation procedures based on at least one of the calculated foreign matter defect coefficient for each action and for each part, and the operation time for each action; and
selecting an operation procedure having a maximum or minimum calculated maintenance operability index.

### REFERENCE SIGNS LIST

1: maintenance operation procedure
110: input unit
111: operation definition information input unit
112: foreign matter source information input unit
113: conversion coefficient input unit
120: display unit
121: calculation result information display unit
130: processing unit
131: foreign matter defect coefficient unit
132: foreign matter diffusion unit
133: maintenance operability index unit
134: maintenance operation generation unit
140: storage unit
141: operation definition information
1411: operation procedure ID
1412: operation ID
1413: operation time
1414: positional relationship information
1415: cleaning part (part for which foreign matter adhesion index is evaluated)
1416: foreign matter source
1417: foreign matter diffusion destination
1418: foreign matter diffusion source
142: foreign matter source information
1421: foreign matter adhesion index
1422: contact foreign matter diffusion rate
1423: above foreign matter diffusion rate
143: calculation result information
1431: foreign matter defect coefficient
1432: total maintenance time
1433: rank of procedure
1434: total foreign matter defect coefficient
1435: total operation time
200: reaction container portion of equipment
201: lower container
201A: first action in assembly operation of lower container
202: upper container
202B: first action in assembly operation of upper container
202
202C: second action in assembly operation of upper container 202
203: screw
203D: first action in assembly operation of screw 203
203E: second action in assembly operation of screw 203
204: cylindrical part
204F: first action in assembly operation of cylindrical part 204
204G: second action in assembly operation of cylindrical part 204
205: lid
205H: first action in assembly operation of lid 205
205J: second action in assembly operation of lid 205
206: internal space of reaction container portion 200
207: cleaning surface of reaction container portion 200
208: hand of operator who assembles reaction container portion 200, which is foreign matter source
209: face of operator who assembles reaction container portion 200, which is foreign matter source
300: directed graph for calculating assembly sequence of reaction container portions 200
301: assembly sequence list of reaction container portion 200
901: first assembly procedure of reaction container portion 200
902: second assembly procedure of reaction container portion 200
S101: operation definition information reading processing
S102: foreign matter defect coefficient calculation processing
S103: foreign matter diffusion calculation processing
S104: maintenance operability index calculation processing
S105: operation ID selection processing

1. An evaluation system of an operation including a plurality of steps on an object having a surface for which a predetermined level of cleanliness is required, wherein
the evaluation includes calculating a total sum of a sum of a plurality of operation times required for the respective plurality of steps, and a product of the sum of the operation times and a coefficient obtained in advance indicating a degree of an occurrence of a defect due to a foreign matter deteriorating the level of cleanliness of the surface in the plurality of steps after the operation based on an evaluation result on a degree of diffusion or adhesion of the foreign matter, and
the calculated result is displayed or notified on a display.

2. The evaluation system of the operation according to claim 1, wherein
the operation is to construct the object by assembling or attaching a plurality of parts, at least one of which includes the surface for which the predetermined level of cleanliness is required, or to disassemble the object into at least one of the plurality of parts.

3. The evaluation system of the operation according to claim 2, wherein
the evaluation includes calculating, for each of the plurality of steps, the coefficient indicating the degree of the occurrence of the defect due to the foreign matter after the operation, using information obtained in advance on a relative position of the part and a foreign matter source including at least one portion of a body of an operator.

4. The evaluation system of the operation according to claim 3, wherein
the evaluation includes calculating, for each of the plurality of steps, the coefficient indicating the degree of the occurrence of the defect due to the foreign matter after the operation, based on a product of the degree of adhesion of the foreign matter from the foreign matter source to the part targeted in the step and the degree of diffusion of the foreign matter in the step.

5. The evaluation system of the operation according to claim 4, wherein
the evaluation includes calculating a sum of the product of the degree of adhesion and the degree of diffusion of the foreign matter for each of the plurality of steps, as the coefficient indicating the degree of the occurrence of the defect due to the foreign matter after the operation.

6. The evaluation system of the operation according to claim 4, wherein
the evaluation includes calculating the coefficient indicating the degree of the occurrence of the defect due to the foreign matter after the operation by using a coefficient obtained in advance indicating a correlation between a value of the sum of the product of the degree of adhesion and the degree of diffusion of the foreign matter for each of the plurality of steps and the degree of the occurrence of the defect due to the foreign matter after the operation.

7. The evaluation system of the operation according to claim 1, wherein
among the plurality of operations for the object which achieve the same result, one operation in which the total sum is the smallest is displayed or notified.
